(19) United States
(12) Patent Application Publication
(10) Pub. No.: US 2013/0066148 A1
(43) Pub. Date: Mar. 14, 2013

(54) METHOD OF AUGMENTING GLANS

(71) Applicants: Kang Seon CHO, Seoul (KR); Dong Soo YU, Seoul (KR); Dae Yul YANG, Seoul (KR)

(72) Inventors: Kang Seon CHO, Seoul (KR); Dong Soo YU, Seoul (KR); Dae Yul YANG, Seoul (KR)

(21) Appl. No.: 13/230,188

(22) Filed: Sep. 12, 2011

(51) Int. Cl. A61F 5/00

(52) U.S. Cl. 600/40

(57) ABSTRACT

Disclosed is a method of augmenting the glans that can safely and naturally expand the glans without causing problems such as an unnatural rough or shiny surface of the glans and the severe side effects of epidermal necrosis, which are caused by the conventional method of augmenting the glans. The method includes inserting an injector needle for injecting a filler into the central area of the lamina propria through the peripheral area of the ostium urethrae externum of the glans, slowly injecting the filler while checking with visual and tactual senses that the glans tissue where the filler is injected swells up and becomes tight and pressure resistance occurs and is maintained during injection of the filler, and slowly injecting the filler whereby the filler is uniformly spread by changing the depth and the direction of the inserted injector needle to expand half the glans area with a one-time injection.

### BACKGROUND OF THE INVENTION

The present invention relates to a method of augmenting the glans, and more particularly, to a method of augmenting the glans safely in a natural shape by injecting a filler containing a collagen-stimulating function deep into the central area of the lamina propria of the glans so as to solve problems such as an unnatural rough or shiny surface of the glans and severe side effects such as tissue necrosis, which is caused by the use of the conventional method of augmenting the glans.

Since penile augmentation that grafts dermofat in the subcutis of the phallus started in the 1990s, the demand for penile augmentation has been growing and a demand for augmentation of the glans has also been growing. However, it is difficult to find an operating method that involves augmenting the glans which has been verified as effective and safe.

As a method of augmenting the phallus, there is a method called “dermofat graft augmentation phalloplasty” in which the phallus is simultaneously increased in length and girth by grafting the dermofat tissue. In addition to this method, methods of grafting a commercially available dermal tissue, which has been obtained by processing homogeneous or heterogeneous skin tissue, in the subcutis of the phallus were commonly used in the 2000s. A method of injecting a filler has been recently introduced and increasingly used.

In particular, the disharmony in size of the phallus and the glans resulting from augmentation of the phallus further increases the necessity of augmenting the glans, but it is still difficult to find a safe and effective method of augmenting the glans.

FIG. 1 shows photographs taken after injecting hyaluronic acid right under the epithelium of the glans as a conventional method of augmenting the glans, which only results in protruding the epithelium of the glans (the superficial layer of the lamina propria).

The procedure has been widely used in some nations including Korea, but it has shown numerous serious side effects in terms of effect and safety. When the hyaluronic acid is not uniformly injected under the epithelium of the glans, the surface of the glans becomes rough and untidy, and even when it is uniformly injected under the epithelium, the surface becomes thin and shiny and thus the skin becomes unnatural. In addition, and the hyaluronic acid is rapidly absorbed such that the expansion effect is reduced and the surface of the glans becomes rough, thereby causing an unnatural shape of the glans. Numerous problems, which have been found so far, also include serious side-effects such as epidermal necrosis.

Although some surgeons have attempted a method of making a space between the corpus cavernosum penis and the corpus spongiosum glans and inserting dermofat tissue, a commercially available dermis or a silicon implant into the space, the method has a serious problem in that the space between the corpus cavernosum penis and the corpus spongiosum glans is not sufficient and the glans augmentation effect is poor, as well as other side effects. These include difficulties in making the surgical space between the corpus cavernosum penis and the corpus spongiosum glans, bleeding, inflammation, and perforation of the glans.

The main reasons that a safe and effective method of augmenting the glans has not been established yet include the unclear anatomic structure of the glans and poor understanding of the anatomic structure, the limitation in surgical operation ability, and the lack of suitable grafting materials.

### SUMMARY OF THE INVENTION

The present invention has been made in an effort to provide a new method of augmenting the glans that can safely augment the glans in a natural shape while keeping the original shape of the glans.

The present invention has been made in an effort to provide a new method of augmenting the glans that can additionally augment the glans by reinjection of a filler. This method keeps the natural shape of the glans without deforming the surface of the glans even when the glans augmentation effect is reduced due to dissolution and absorption of the components of the filler.

An exemplary embodiment of the present invention provides a method of augmenting the glans, characterized in that a filler is injected into the central area of the lamina propria of the glans. In detail, the filler has a high collagen-stimulating function and the filler is injected through an injector needle positioned aslant in the central area of the lamina propria. The needle may be inserted aslant into the central area of the lamina propria through the peripheral area of the ostium urethrae externum of the glans, where the lamina propria forms the thickest layer. The filler may be injected while checking with visual and tactual senses during injection of the filler that the glans tissue where the filler is injected swells up and becomes tight and the pressure resistance occurs and is maintained. Further, the filler may be injected such that the filler uniformly spreads by changing the depth and the direction of the injector needle inserted in the glans to expand half the glans area with a one-time injection.

The present invention has the following advantages with the configuration described above.

The present invention provides a method of injecting a filler deep into the central area of the lamina propria of the glans. The method of augmenting the glans is a method of significantly expanding the lamina propria without unnaturally protruding the epithelial portion of the glans, which occurs when a hyaluronic acid filler is injected into the lamina propria right under the surface of the glans, and without causing the filler to flow into the corpus spongiosum, which make it possible to safely augment the glans in a natural shape while keeping the relative original shape of the glans.

### BRIEF DESCRIPTION OF THE DRAWINGS

FIG. 1 is a photograph of the shape of the glans after injecting a filler according to a conventional method of augmenting the glans.

FIG. 2 is a photograph showing three layers of the glans.

FIG. 3 is a photograph of the shape of the glans after injecting a filler according to a method of augmenting the glans consistent with the present invention.

FIG. 4 is a diagram showing a method of augmenting the glans according to an exemplary embodiment of the present invention.

### DETAILED DESCRIPTION OF THE INVENTION

The corpus cavernosum of the penile shaft is encased with a distinct anatomic structure, the tunica albuginea (the tough fibrous layer of connective tissue that surrounds the corpora cavernosum of the penis), such that it is definitely discriminated from the outer tissue. Therefore, a method of engrafting an implant or injecting a suitable filler into the outside of the tunica albuginea encasing the corpus cavernosum through an operation is useful as a means for augmenting the phallus.

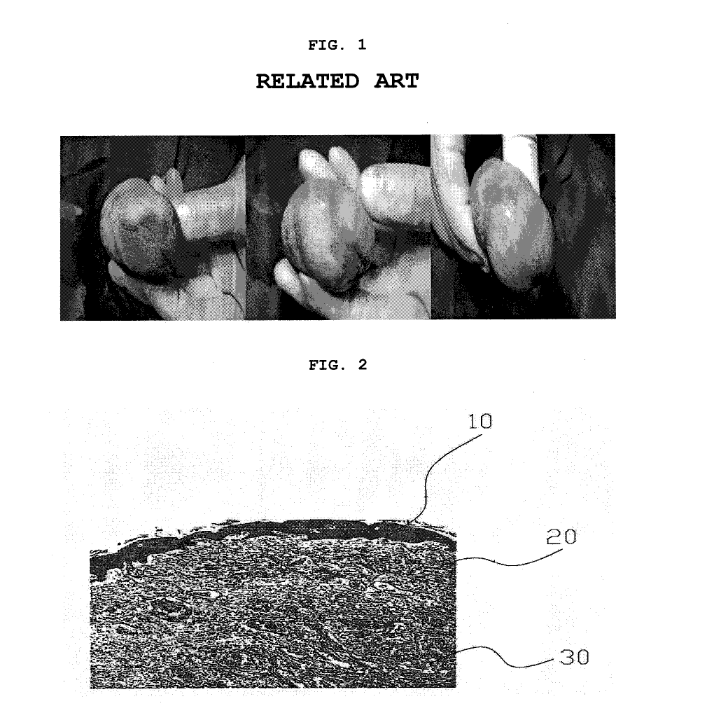

However, there is no distinct anatomic structure corresponding to the tunica albuginea of the penile shaft in the glans and there is also no dermis layer, which exists at any portion of the skin of the whole body. However, referring to the photograph showing three layers of the glans in FIG. 2, the glans has a epithelium layer composed of the non-keratinized stratified squamous epithelium having the same thickness as that of five or six cells, and a lamina propria 20 (the prolongation of the loose connective tissue of the foreskin submucosa or lamina propria, forming a sort of aponeurosis encasing and separating the corpus spongiosum from the glans epithelium) is formed under the epithelium layer to encase the innermost corpus spongiosum 30 and separate the outermost stratified squamous epithelium from the innermost corpus spongiosum. In this case, the corpus spongiosum does not have an anatomically definite boundary with respect to the outer tissue encasing the corpus spongiosum.

The corpus spongiosum is a main tissue of the glans and a blood vessel tissue composed of the specialized venous sinuses and is connected with the general venous system. Therefore, when the filler is injected into the corpus spongiosum, the filler may be injected into the general venous system, which leads to a corresponding serious risk.

Up to now, it was prohibited to inject a filler deep into the central area of the glans, due to the risk that may be caused when the filler is injected into the corpus spongiosum and the unclear anatomical/structural features of the glans which are difficult to understand.

However, the lamina propria is a loose connective fibrous tissue that is not directly connected with the corpus spongiosum and the thickness of the lamina propria varies, depending on the position, such as 1 mm at the corona and 2.5 mm around the ostium urethrae externum.

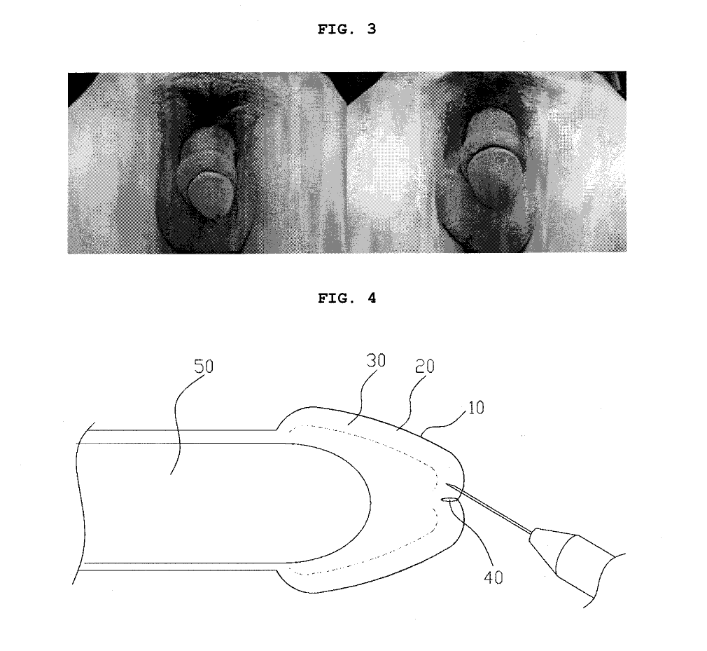

The present invention provides a method of significantly expanding the lamina propria by injecting a filler deep into the central area of the lamina propria of the glans without causing the surface of the glans to protrude and also without causing the filler to flow into the corpus spongiosum. Unlike when the surface of the glans becomes protruded by injecting the hyaluronic acid under the epithelium of the glans, when the filler is injected into the central area of the lamina propria to uniformly expand the lamina propria, the glans is augmented in a natural shape while keeping the relative original shape (FIG. 3). Further, even when the expansion effect is reduced as the components of the filler are dissolved and absorbed, the shape of the glans does not change such that the glans can be additionally augmented by reinjection of the filler while keeping the natural shape of the glans. This result is the main factor that significantly increases the satisfaction of the client.

The filler is preferably a substance that flows as little as possible into the corpus spongiosum after injection into the lamina propria. This is because the filler can cause serious side effects if even a small amount of the filler is injected into the general venous system. Therefore, a filler having a high collagen-stimulating function, such as a PMMA or dextranomer, is preferred when compared to a filler having no collagen-stimulating function such as the hyaluronic acid. This is because a filler including PMMA or dextranomer as the main component, for example, and a filler having a high collagen-stimulating function, such as LIPEN (trade name), has functions which prevent the filler components from flowing around after injection and help the glans augmentation effect remain for a longer time.

The technique of uniformly injecting the filler into the lamina propria is very important for one exemplary embodiment of the present invention in which the glans is augmented in a natural shape by injecting the filler deep into the central area of the lamina propria.

When a filler is injected into the lamina propria but largely injected under the epithelium of the glans like the technique known in the art, the epithelium of the glans is separated from the lamina propria and a corresponding epithelial portion of the glans immediately protrudes, which is one of the above-mentioned problems that the present invention intends to solve. Further, it is necessary to avoid a dangerous situation in which the filler is injected into the corpus spongiosum deeper than the lamina propria, in which case it is difficult to feel pressure resistance during injection of the filler and it is also difficult to recognize immediate tightness and expansion of the glans with visual or tactual sense.

The method of augmenting the glans according to the present invention will be described with reference to FIG. 4.

A preferable method includes slowly injecting a filler by inserting a needle at a tilted angle from a portion 40 around the ostium urethrae externum where the lamina propria forms the thickest layer to the central area of the lamina propria such that the filler spreads around with pressure resistance during injection. It is necessary to keep feeling the pressure resistance during the injection of the filler. In this case, by checking that the filler is slowly injected it can be confirmed with visual and tactual senses that the filler accurately spreads out into the lamina propria 20 and that the corresponding portion gradually become tight and slowly swells up. Sometimes, it is possible to complete the procedure for half the glans area with a one-time injection whereby the filler is uniformly injected in the lamina propria of the glans by slightly changing the depth and the direction of the inserted needle, and then to complete the other half in the same way.

1. A method of augmenting the glans, comprising:
injecting a filler into the central area of the lamina propria of the glans.

2. The method of claim 1, wherein the filler has a high collagen-stimulating function.

3. A method of augmenting the glans, comprising:
inserting an injector needle for injecting a filler into a central area of the lamina propria through a peripheral area of the ostium urethrae externum of the glans;
slowly injecting the filler while checking with visual and tactual senses that the glans tissue where the filler is injected swells up and becomes tight and pressure resistance occurs and is maintained during injection of the filler; and
slowly injecting the filler such that the filler uniformly spreads by changing depth and direction of the inserted injector needle to expand half the glans area with a one-time injection.

4. The method of claim 3, wherein the filler has a high collagen-stimulating function.
